(19) United States
(12) Patent Application Publication
(10) Pub. No.: US 2017/0258593 A1
(43) Pub. Date: Sep. 14, 2017

(54) CARDIAC ASSISTANCE DEVICE

(71) Applicants: David Good, Alamogordo, NM (US); Rafael Alvergue, Lafayette, LA (US)

(72) Inventors: David Good, Alamogordo, NM (US); Rafael Alvergue, Lafayette, LA (US)

(21) Appl. No.: 15/065,577

(22) Filed: Mar. 9, 2016

(51) Int. Cl. A61F 2/24

(52) U.S. Cl. CPC A61F 2/2478

(57) ABSTRACT

A device for assisting the operation of a natural heart is provided. A supporting jacket shaped to surround at least a portion of a heart has an expandable membrane attached to the inside wall of the jacket so that the membrane faces the heart. An inflatable cavity is formed between the jacket and the membrane. The cavity is connected to an expandable fluid reservoir via a length of flexible tubing. Pumps are used to pump fluid back and forth between the cavity and the reservoir. The cavity if inflated as the heart contract to aid the heart in pumping blood. The cavity is deflated as the heart relaxes to allow the heart to refill with blood. The cycle of pumping fluid is synchronized with the cardiac cycle.

### FIELD OF THE INVENTION

A preferred embodiment of the invention refers to a cardiac assistance device and a method of operating the device.

### BACKGROUND

It is estimated that over 5 million people in the United States currently suffer from heart failure. Over 500,000 new cases of heart failure are diagnosed every year, and about half of people who develop heart failure die within five years of diagnosis. When heart failure occurs, the heart continues to function but becomes weaker than a healthy heart. The weakened heart is unable to pump a sufficient amount of blood and oxygen throughout the body to support other organs. Heart failure may be caused or worsened by a variety of conditions including coronary artery disease, heart attacks, or high blood pressure, among other conditions.

A ventricular assist device is an apparatus used to partially or completely replace the function of a failing heart. The most common type is a left ventricular assist device (LVAC). Ventricular assist devices may be used in some cases as a permanent treatment for heart failure or as a bridge to heart transplantation. One common type of ventricular assist device connects directly to the circulatory system and utilizes blood as the working fluid. Such devices typically employ a pump used to aid the heart in pumping blood throughout the body. Due to direct contact with blood, such devices may cause damage to the blood, and hemolysis is recognized as a major complication.

Other known ventricular assist devices do not have direct contact with the blood but instead act externally on the heart muscles to assist in the contraction of the heart. A number of problems are associated with such devices engaging externally with the muscles of the heart. Some known systems are overly complex, utilizing numerous individual parts. Other known systems may not have the ability to easily adjust to the changing dimensions of a failing heart as the heart's function improves or deteriorates over time. Additionally, some known systems may hinder the heart's normal functioning in the event of a system failure by restricting the heart's diastolic expansion and refilling of blood.

Accordingly, a need exists in the art for a cardiac assistance device for partially or completely replacing the function of a failing heart. In addition, a need exists in the art for a cardiac assistance device that is not overly complex and utilizes a minimal number of parts. Further, a need exists in the art for a cardiac assistance device that can automatically adjust to changing heart dimensions and that will not hinder the functioning of the heart in the event of system failure.

### SUMMARY

A preferred embodiment of the invention is directed to a device for assisting the operation of a natural heart by applying external pressure on a ventricle of the heart. The device applies pressure in a pulsed manner so that it at least partially replaces the pumping operation of the heart. By taking over some of the work performed by the heart, the device may aid in the recovery of a failing heart.

In one aspect of the present invention, a ventricular assist device comprises a supporting jacket configured for surrounding at least a portion of a heart and an expandable membrane attached to the jacket. The membrane faces inwardly toward the heart and forms an inflatable cavity between the jacket and the membrane. The device further comprises an expandable fluid reservoir and at least one pump configured for pumping fluid back and forth between the reservoir and the cavity, which is located directly adjacent to the heart. During systolic contraction of the ventricles, fluid is pumped into the cavity to inflate the cavity and exert external pressure on the ventricle, thereby assisting the heart in contraction. During diastolic relaxation and filling of the ventricles, fluid is pumped out of the cavity and into the reservoir so that the heart can expand to its natural volume at diastole. The simple back-and-forth pumping configuration of the system minimizes the number of individual parts, which is advantageous for space and weight considerations, and allows the system to be integrated into the body with minimal invasiveness.

In one embodiment, the device comprises a single bidirectional pump that pumps fluid between the reservoir and the cavity in both directions. In other embodiments, the device may comprise a plurality of pumps in a parallel configuration such that separate pumps are utilized for pumping fluid from the reservoir to the cavity and from the cavity to the reservoir, respectively. In each case, a common line fluidly connects the reservoir and the cavity. The working fluid used in the system is preferably water.

In a preferred embodiment, the device further comprises a plurality of flow control valves configured for controlling the direction of flow between the cavity and the reservoir. Flow control valves are preferably located on the discharge side of each pump in a parallel configuration. During systolic contraction, a first pump configured for pumping fluid into the cavity is actuated, and a control valve located at the pump discharge is opened. A second pump configured for pumping fluid from the cavity into the reservoir is concurrently deactivated, and a control valve located at the discharge of the second pump is closed. During diastolic relaxation and filling of the ventricles, the second pump is actuated and the first pump is deactivated, and each associated control valve switches between the open and closed positions.

In another embodiment, additional control valves may be located on pump bypass lines. In this embodiment, each pump has two bypass lines configured such that a single pump that pumps in only one direction can be used to pump fluid between the cavity and the reservoir in both directions by utilizing one bypass line for each flow direction, respectively. When utilizing a two-pump system for normal operation, the control valves on the bypass lines remain closed. In the event of a pump failure, the second pump can be used to maintain normal operation by alternately opening and closing the valves on each bypass line, respectively.

The fluid reservoir is made of an expandable material such that the reservoir can expand and contract according to the quantity of fluid contained within the reservoir at a given time. The expandable nature of the reservoir provides the system with added flexibility with respect to the amount of fluid that can be contained within the system and the range of pressures that can be produced by the system for hearts experiencing varying degrees of heart failure. The added flexibility also allows the system to easily adjust over time to changing heart dimensions as the condition of the heart improves or deteriorates with time. Additionally, in the event of a system failure, the expandable capacity of the reservoir minimizes backpressure on the heart and thus allows the heart to expand to its maximum volume so that the heart operates normally without resistance from the fluid contained within the system.

The device further comprises a central control system located within the body containing the heart. The control system is operably connected to all pumps and control valves. The control system is responsible for activating and deactivating pumps and switching control valves between the open and closed positions so that the pumping operation of the device is synchronized with the cardiac cycle of the heart. The control system can also vary the speed of rotation of each pump to maintain predefined systolic and diastolic pressures within the cavity. In the event that a pump fails, the control system may detect the pump failure and automatically adjust the system to maintain normal operation using only a second pump.

Accordingly, one object of the present invention is to provide a cardiac assistance device for partially or completely replacing the function of a failing heart.

Another object of the present invention is to provide a cardiac assist device that pumps fluid back and forth between a fluid reservoir and an inflatable cavity adjacent to a ventricle of a heart to assist the contraction of the heart for pumping blood throughout the body.

Another object of the present invention is to provide a cardiac assistance device that can be seamlessly integrated into the body.

Another object of the present invention is to provide a cardiac assistance device that can easily adjust over time to changing heart dimensions as the condition of the heart improves or deteriorates with time.

Another object of the present invention is to provide a cardiac assistance device that will not hinder the functioning of the heart in the event of system failure.

Furthermore, an object of the present invention is to provide a method of operating such a device.

### DESCRIPTION OF THE DRAWINGS

These and other features, aspects, and advantages of the present invention will become better understood with regard to the following description, appended claims, and accompanying drawings where:

FIG. 1 shows a schematic view of a cardiac assistance device in accordance with the present invention, including a control system.

FIG. 2 shows a partial view of a cardiac assistance device fitted to a heart in accordance with the present invention.

FIG. 3 shows a schematic view of a cardiac assistance device fitted to a heart in accordance with the present invention

FIG. 4 shows a schematic view of one embodiment of pumps, valves, and lines for controlling fluid flow of the device depicted in FIG. 1.

### DETAILED DESCRIPTION

In the Summary above and in this Detailed Description, and the claims below, and in the accompanying drawings, reference is made to particular features, including method steps, of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with/or in the context of other particular aspects of the embodiments of the invention, and in the invention generally.

The term “comprises” and grammatical equivalents thereof are used herein to mean that other components, ingredients, steps, etc. are optionally present. For example, an article “comprising” components A, B, and C can contain only components A, B, and C, or can contain not only components A, B, and C, but also one or more other components.

Where reference is made herein to a method comprising two or more defined steps, the defined steps can be carried out in any order or simultaneously (except where the context excludes that possibility), and the method can include one or more other steps which are carried out before any of the defined steps, between two of the defined steps, or after all the defined steps (except where the context excludes that possibility).

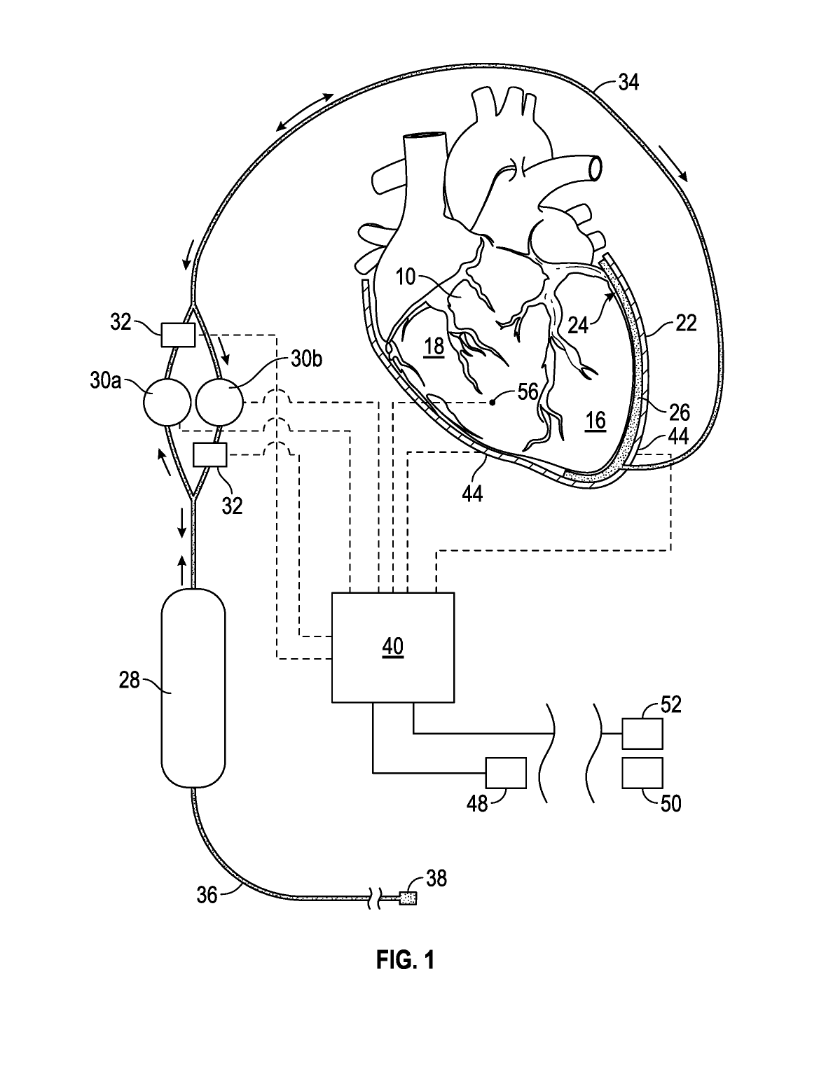

Turning now to the drawings, FIG. 1 shows an overview of a cardiac assistance device having a supporting jacket 22 configured to surround at least a portion of a heart 10. The device assists the operation of the natural heart 10 by applying external pressure on a ventricle 16, 18 of the heart 10. The device applies pressure in a pulsed manner so that it at least partially replaces the pumping operation of the heart. By taking over some of the work performed by the heart, the device may aid in the recovery of a failing heart.

In one aspect, the present invention comprises the supporting jacket 22 and an expandable membrane 24 attached to the jacket 22. The membrane 24 is configured to face inwardly toward the heart 10 and forms an inflatable cavity 26 between the jacket 22 and the membrane 24. The jacket 22 has a generally concave shape configured to conform to the contours of a particular heart 10. A unique jacket 22 is preferably formed for each individual patient to better adapt to each patient's heart. The three-dimensional shape of the jacket 22 can be determined based on cardiac data obtained from each patient. The data may be obtained by MRI, CT scan, or similar methods known in the art. The data may then be used to produce the three-dimensional jacket 22 by 3D printing, injection molding, or similar methods known in the art.

The jacket 22 is made of a relatively stiff material such that the jacket 22 substantially retains its shape during normal operation of the cardiac assistance device. The jacket 22 is preferably capable of bending somewhat if compressive forces are applied to the jacket 22, but the jacket 22 should substantially return to its original shape.

To reduce friction on the heart 10 during normal operation, the inside wall of the supporting jacket 22 preferably has a coating comprising a low-friction material.

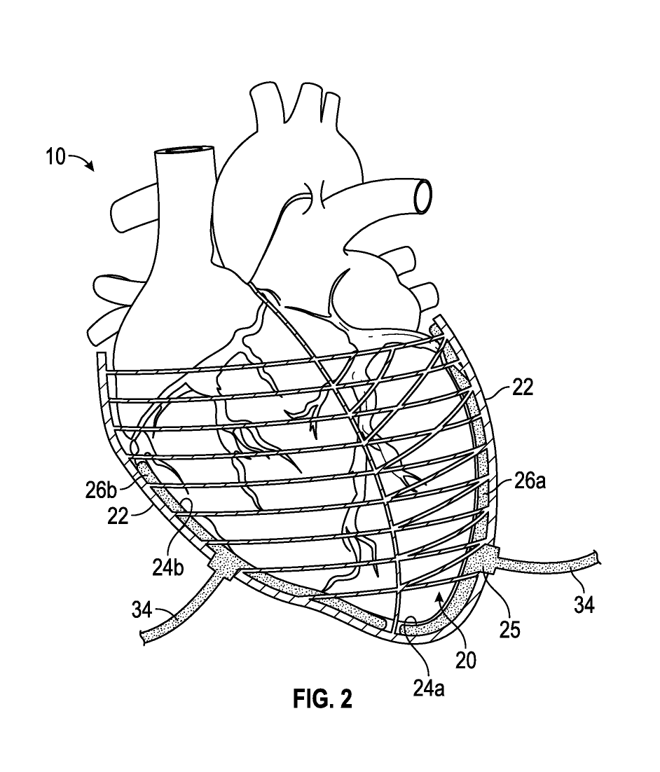

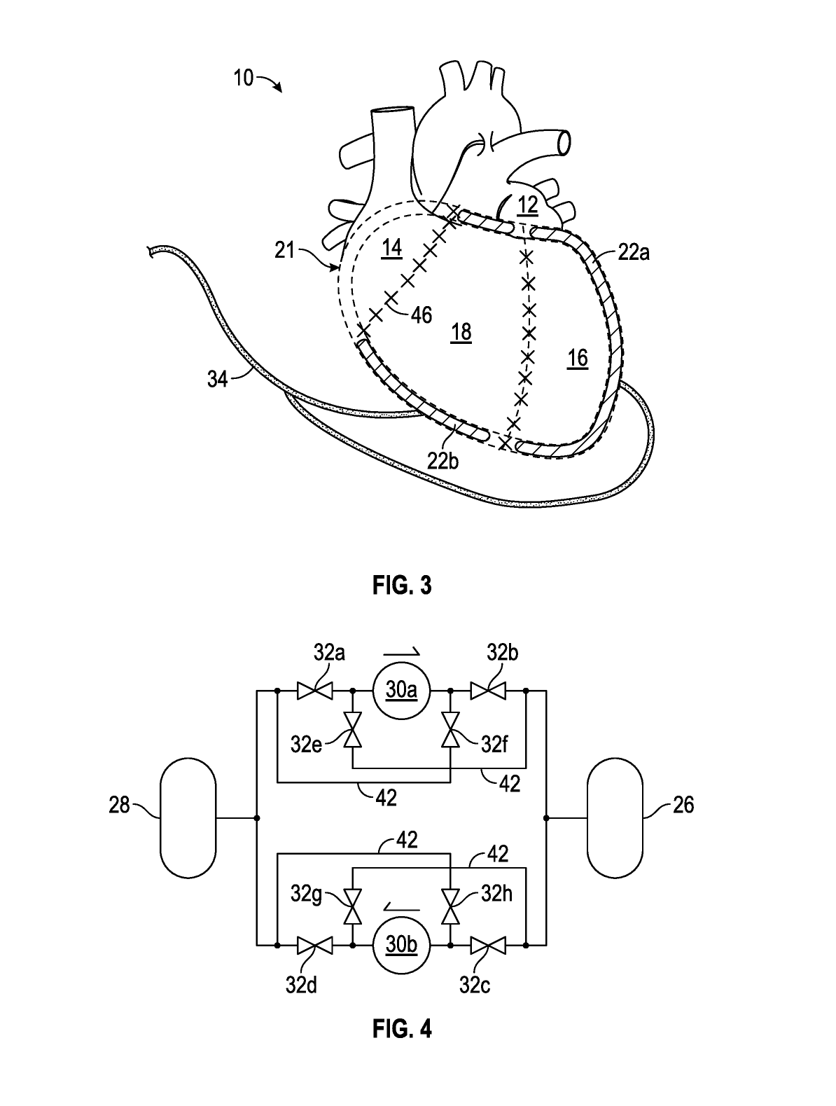

In one embodiment, as shown in FIGS. 1-2, the jacket 22 is configured to surround a substantial portion of both the left ventricle 16 and the right ventricle 18 such that the device may support contraction of both ventricles. In some cases, a patient may require only left ventricular assistance. In such cases, the jacket 22 may be configured to surround only the left ventricle 16 to minimize the size of the jacket 22. In a preferred embodiment, the jacket 22 is attached to the pericardium 21, preferably by stitching the jacket 22 to the pericardium 21. The jacket 22 preferably has a plurality of anchor points that can be used to stitch the jacket 22 to the pericardium 21. The jacket 22 is preferably stitched to the inside of the pericardium 21, as shown in FIG. 3, to maintain constant orientation and prevent migration throughout the cardiac cycle. The jacket 22 may alternatively be stitched to the outside of the pericardium 21.

In cases in which both left and right ventricular assistance is required, the device preferably comprises a separate supporting jacket for each respective ventricle. As shown in FIG. 3, a first jacket 22a is configured to surround at least a portion of the left ventricle 16, and a second jacket 22b is configured to surround at least a portion of the right ventricle 18. In a preferred embodiment, both jackets 22a, 22b are stitched to the inside of the pericardium 21 and additionally stitched to each other as indicated by the stitch marks 46 shown in FIG. 3. The joined jackets 22a, 22b form a continuous unit that surrounds a substantial portion of both ventricles 16, 18, but does not generally surround the portion of the heart 10 comprising the left atrium 12 or the right atrium 14. Utilizing two separate jackets 22a, 22b allows for easier fitting of the device around a patient's heart 10.

The expandable membrane 24 is attached to the interior wall of the supporting jacket 22 along the periphery of the membrane 24 so that the membrane 24 faces inwardly toward the heart 10, preferably in direct contact with the myocardium. The membrane 24 forms an inflatable, expandable cavity 26 between the jacket 22 and the membrane 24. The membrane 24 shown in FIG. 1 is configured to contact the left ventricle 16. In cases in which both left and right ventricular assistance is required, two separate membranes are preferably utilized. FIG. 2 shows a partial view of a cardiac assistance device having two membranes 24a, 24b. In a preferred embodiment, the first membrane 24a is attached to the first supporting jacket 22a to form a first cavity 26a. The second membrane 24b is attached to the second jacket 22b to form a second cavity 26b. Each cavity, 26a and 26b, is configured to apply pressure to each ventricle, 16 and 18, respectively, during systolic contraction of the ventricles.

As shown in FIG. 1, the device further comprises an expandable fluid reservoir 28 and at least one pump configured for pumping fluid back and forth between the reservoir 28 and the inflatable cavity 26. In a preferred embodiment, two pumps 30a, 30b are arranged in parallel for pumping the fluid. The fluid is pumped back and forth via a common line 34 fluidly connecting the reservoir 28 to the cavity 26. Flow control valves 32 are located at the discharge of each pump 30a, 30b. Additional valves may optionally be located on the inlet side of each pump 30a, 30b, as shown in FIG. 4. During systolic contraction of the left ventricle 16, a first pump 30a is actuated, and the control valve 32 at the pump discharge is simultaneously opened. A second pump 30b is concurrently deactivated as the control valve 32 at the discharge of the second pump 30b is closed. This combination of actions pumps fluid from the reservoir 28 into the cavity 26 to inflate the cavity 26 and exert external pressure on the left ventricle 16, with maximum pressure in the cavity 26 achieved at systole. Inflation of the cavity 26 assists the heart 10 in contracting, thereby aiding the heart in pumping blood throughout the body. Conversely, during diastolic relaxation and filling of the left ventricle 16, the second pump 30b is actuated, and the control valve 32 at the discharge of the second pump 30b is simultaneously opened. The first pump 30a is concurrently deactivated as the control valve 32 at the first pump discharge is closed. This combination of actions pumps fluid from the cavity 26 to the reservoir 28, which causes deflation of the cavity 26, to relieve pressure on the ventricle 16. Deflation of the cavity 26 allows the ventricle 16 to expand to its natural volume at diastole. This process is repeated in synchronization with the cardiac cycle.

FIG. 4 shows a schematic diagram of an alternative embodiment also utilizing the same two pumps 30a, 30b in parallel. For ease of illustration, the cavity 26 and reservoir 28 are represented in FIG. 4 by vessels 26 and 28, respectively. In this embodiment, each pump 30a, 30b is unidirectional, i.e., is capable of pumping in only one direction. The arrows shown in FIG. 4 indicate the pumping direction of each pump. In addition, each pump 30a, 30b has two bypass lines 42 configured such that each unidirectional pump 30a, 30b can be used to pump fluid between the cavity 26 and the reservoir 28 in both directions. This configuration allows a single pump to be used to maintain normal operation in the event that one of the pumps fails. When both pumps 30a, 30b are operating normally, the valves 32e, 32f, 32g, 32h on the bypass lines 42 all remain closed. In this case, valves 32a and 32b are open when pump 30a is activated and closed when pump 30a is deactivated. Similarly, valves 32c and 32d are open when pump 30b is activated and closed when pump 30b is deactivated. In the event that pump 30b, for instance, should fail, pump 30a can be used to maintain normal operation of the device. In this example, valves 32c, 32d, 32g, and 32h are first closed for the duration of the failure of pump 30b. Next, to pump fluid from the reservoir 28 to the cavity 26 during systolic contraction, valves 32a and 32b are opened and valves 32e and 32f are closed. To pump fluid from the cavity 26 to the reservoir 28 during diastolic relaxing, valves 32e and 32f are opened and valves 32a and 32b are closed. In the event that pump 30a should fail, pump 30b and its associated bypass lines 42 and valves 32c, 32d, 32g, 32h may be operated in a similar manner to maintain normal operation of the system. Bypass lines arranged as shown in FIG. 4 provide redundancy in the design to ensure normal operation of the device at all times.

In another embodiment, the device may comprise a bidirectional pump. The pump is capable of changing the direction of rotation of its impeller such that a single pump can be used for pumping the fluid in both directions. The pump changes the flow direction of the fluid in synchronization with the cardiac cycle to inflate the cavity 26 during systolic contraction and deflate the cavity 26 during diastolic refilling of the ventricles in the manner previously described. The device may optionally comprise a second bidirectional pump to serve as a backup pump. The second bidirectional pump is in parallel with the first pump and is activated only in the event that the first pump fails. Each pump has a control valve located at the pump discharge. In normal operation using only the first pump, the valve on the first pump discharge remains open at all times, and the valve on the second pump discharge remains closed at all times. If the second pump is activated, the valve on the first pump discharge is closed and the valve on the second pump discharge is opened for the duration of the operation of the second pump.

The back-and-forth pumping design of the system as described herein minimizes the complexity of the system and reduces the weight and the amount of space required of the system. These advantages help to reduce the invasiveness of the medical procedure of integrating the device into the body.

In a preferred embodiment, the one or more pumps 30a, 30b are magnetic drive pumps, wherein the pump impeller is magnetically couple to the pump drive shaft. Magnetic pumps reduce the probability of fluid leakage from the system and typically require less maintenance than conventional centrifugal pumps, though any suitable pump may be utilized with the present invention.

The fluid reservoir 28 is made of an expandable material such that the reservoir 28 can expand and contract according to the quantity of fluid contained within the reservoir 28 at a given time. The reservoir may be made of any suitable expandable material, which may be the same material as the expandable membrane 24 attached to the supporting jacket 22. The reservoir 28 is preferably disposed within the body containing the heart 10, preferably within the thoracic cavity. The reservoir 28 is fluidly connected to the inflatable cavity 26 adjacent the heart 10 by a length of flexible tubing 34. The tubing 34 is attached to the reservoir 28 at one end and to an aperture 25 in the supporting jacket 22 at the opposite end. For the cavity 26a assisting the left ventricle 16, the aperture 25 is preferably configured to be located adjacent to the apex 20 of the heart 10, as shown in FIGS. 1-2. In embodiments utilizing a second cavity 26b for assisting the right ventricle 18, the tubing 34 splits and connects to a separate aperture adjacent to the right ventricle 18, as shown in FIGS. 2-3. In alternative embodiments, one or both cavities 26a, 26b may be fluidly connected to the reservoir 28 through a plurality of apertures in the supporting jacket 22.

As shown in FIG. 1, the device further comprises a central control system 40 operably connected to all pumps 30a, 30b and control valves 32 and programmed to control the pumps and valves. The control system 40 is preferably located subcutaneously and comprises a valve control system configured for independently opening and closing each valve 32. For instance, the valve control system is responsible for opening each respective valve 32 at the discharge of pumps 30a and 30b when the corresponding pump is activated and for closing each respective valve 32 at the discharge of pumps 30a and 30b when the corresponding pump is deactivated, as previously discussed.

The control system 40 further comprises a pump control system configured to independently vary the speed of rotation of the pumps 30a and 30b to maintain predefined systolic and diastolic pressures within the cavity 26. The pump control system is in communication with a pressure sensor system comprising pressure sensors 44 that detect the pressure in each of the cavities 26a and 26b. In response to pressure data recorded in the cavity 26, the control system 40 can vary the speed of rotation of pump 30a to adjust the systolic pressure in the cavity 26 to maintain a predefined target pressure. The control system 40 can independently vary the speed of rotation of pump 30b to adjust the diastolic pressure in the cavity 26.

This feature provides a dynamic system that can adjust over time to changing heart dimensions as the condition of the heart improves or deteriorates with time. For instance, if the heart decreases in size due to improved functioning, the control system 40 can increase the speed of rotation of pump 30a to pump a greater quantity of fluid into the cavity 26 and decrease the speed of rotation of pump 30b to remove a lesser quantity of fluid from the cavity 26.

Pump 30b should be sized such that the pump is capable of pulling a partial vacuum on cavities 26a and 26b between the myocardium and the jacket 22. This feature allows for the transmission of forces in diastole to the myocardium, which reduces ventricular filling pressures and thereby improves diastolic function. This effect results in less strenuous filling of the ventricles 16, 18 and can help a failing heart improve its overall functioning over a period of time of using the device.

The device further comprises a fluid refill line 36 connected at one end to the fluid reservoir 28. In a preferred embodiment, as shown in FIG. 1, the refill line 36 is disposed transcutaneously and extends from the intracorporeal fluid reservoir 28 to the outside of the body. The refill line 36 has a refill valve 38 operable from outside the body. The refill valve 38 functions as an interface that can be used to add fluid to or remove fluid from the system. Adding additional fluid to the system causes the reservoir 28 to expand in size and increases the overall pressure within the system. Thus, the expandable nature of the reservoir 28 provides the system with added flexibility with respect to the amount of fluid that can be contained within the system and the range of pressures that can be produced by the system for hearts experiencing varying degrees of heart failure. This feature allows the amount of fluid to be adjusted according to the specific needs of each patient. For instance, if the functioning of the heart decreases over time, additional fluid can be added to the system to increase the overall pressure within the system, which will increase the pressure in the cavity 26 as the cavity is inflated during systolic contraction. The increased cavity pressure causes the membrane 24 to apply greater external pressure on the ventricles, thereby providing greater assistance to the ventricles in pumping blood to the body. Conversely, an improved heart may need less assistance and so fluid can be removed from the system to lower the overall pressure within the system. In a preferred embodiment, the working fluid is water, though any suitable fluid may be utilized, including a gaseous fluid such as air.

The device of the present invention provides proper fail-safe positions and system redundancy in the event of a failure in the system. In the event of a total system failure due, for example, to a loss of power, all valves 32 in the system fail to the open position, which allows fluid to readily flow between the cavity 26 and the reservoir 28. The expandable capacity of the reservoir 28 minimizes backpressure on the ventricles and allows the heart to expand to its maximum size, thereby allowing the heart to operate normally without resistance from the fluid in the cavity 26. Accordingly, the device of the present invention does not appreciably hinder normal heart functioning in the event of a system failure or deactivation of the device.

The central control system 40 further comprises a pump sensor system responsive to a pump failure. In the event of a failure of one of the pumps, the pump sensor system, in communication with the valve control system, is configured for maintaining normal operation of the device during a pump failure. The pump sensor system is responsible for activating a remaining pump that is properly functioning for use in pumping fluid in both directions by utilizing the bypass lines 42 as previously discussed with respect to FIG. 4. The valve control system is responsible for alternately opening and closing the respective valves located on the fluid line 34 and bypass lines 42 as necessary to maintain normal operation.

In a preferred embodiment, the system is powered by a battery 48 contained within the body. The battery 48 can be charged from outside the body through an electric battery charger 50. Preferably, as shown in FIG. 1, the battery 48 powers the central control system 40 via a direct connection to the system 40. Power is then provided to the pumps 30a, 30b via a connection between the pumps 30a, 30b and the central control system 40.

The central control system 40 further comprises a transmitter 52 for transmitting data from the device. The transmitter 52 can be used by an operator of the device, such as a patient's doctor, to program the central control system 40. For instance, the control system 40 can be reprogrammed to change the target values for systolic and diastolic pressure in the cavities 26a, 26b. In addition, the transmitter 52 can provide patient data to the doctor over a period of time. For example, the doctor can monitor changes or trends in systolic and diastolic pressure within the cavities 26a, 26b. This data is recorded by pressure sensors 44 located in each cavity 26a, 26b and is stored by the central control system 40 for later retrieval by the patient's doctor. This information can then be used by the doctor to determine, for instance, whether fluid should be added to or removed from the system and, if so, how much fluid, or whether the control system 40 requires reprogramming. The data may also indicate to the doctor whether a patient's heart functioning is improving with continued use of the device or whether the heart condition is deteriorating, which may guide the doctor in determining future action to best help the patient.

The control system 40 is also responsible for maintaining the timing of the device so that the device works in synch with the cardiac cycle. In the illustrative embodiment shown in FIG. 1, the central control system 40 activates and drives pump 30a during systolic contraction of the heart to pump fluid into the cavity 26. The control system 40 then reverses the fluid flow by deactivating pump 30a and activating pump 30b to quickly pump fluid out of the cavity 26 to allow the heart to relax and expand naturally. The control system is also responsible for alternately opening and closing the appropriate valves 32 throughout the pumping cycle. The timing of the pumping cycle is controlled such that the highest pressure is achieved at systole and the lowest pressure at diastole. The control system 40 may adjust the timing of the pumping cycle as needed based on data obtained by an electrocardiogram (ECG) sensor 56, which is operably connected to the central control system 40 and is preferably located on the right ventricle 18. The ECG sensor 56 measures electrical signals of the heart 10 and transmits this data to the control system 40 to determine the timing of the natural cardiac cycle. The control system 40 is pre-programmed to continuously process this data to determine the proper timing of the pumping cycle in order to synch the pumping cycle with the natural cardiac cycle and to dynamically adjust the timing, if necessary.

It is understood that versions of the invention may come in different forms and embodiments. Additionally, it is understood that one of skill in the art would appreciate these various forms and embodiments as falling within the scope of the invention as disclosed herein.

What is claimed is:

1. A device for assisting the operation of a natural heart, said device comprising:
a. a supporting jacket configured for surrounding at least a portion of a heart;
b. an expandable membrane attached to the jacket so as to form an inflatable cavity between the jacket and the membrane, said membrane configured to face inwardly toward the heart;
c. an expandable fluid reservoir fluidly connected to the cavity; and
d. at least one pump configured for pumping fluid back and forth between the reservoir and the cavity.

2. The device of claim 1, wherein the jacket has an aperture therethrough, wherein the reservoir is fluidly connected to the cavity via a fluid line connected to the reservoir at one end and the aperture at the opposite end.

3. The device of claim 2, wherein the aperture is configured to be located adjacent to the apex of a heart.

4. The device of claim 1, wherein the jacket is configured such that the jacket is attached to the pericardium.

5. The device of claim 4, wherein the jacket is configured such that the jacket is stitched to the inside of the pericardium.

6. The device of claim 1, wherein the jacket is configured for surrounding at least a portion of the left ventricle such that the membrane contacts at least a portion of the left ventricle.

7. The device of claim 1, wherein the at least one pump is a magnetic drive pump.

8. The device of claim 1, wherein the at least one pump is a bidirectional pump.

9. The device of claim 1, wherein the device comprises a plurality of pumps in a parallel configuration, said device further comprising a plurality of flow control valves configured for controlling the direction of flow between the cavity and the reservoir.

10. The device of claim 9, wherein each pump is unidirectional and has two bypass lines configured such that each pump can be used to pump fluid between the cavity and the reservoir in both directions.

11. The device of claim 9, further comprising a valve control system operably connected to the valves, said valve control system configured for independently opening and closing each valve.

12. The device of claim 10, further comprising a valve control system operably connected to the valves, said valve control system configured for independently opening and closing each valve.

13. The device of claim 12, further comprising a pump sensor system responsive to a pump failure and configured for maintaining normal operation of the device during pump failure by activating a single pump and alternately opening and closing valves such that the single activated pump can be used to pump fluid back and forth between the cavity and the reservoir.

14. The device of claim 1, wherein the fluid reservoir is disposed within the body containing the heart, said device further comprising a transcutaneous fluid refill line configured for adding fluid to or removing fluid from the reservoir.

15. The device of claim 1, further comprising a pressure sensor system configured for detecting fluid pressure within the cavity.

16. The device of claim 15, further comprising a pump control system in communication with the pressure sensor system and configured to vary the speed of rotation of the at least one pump to maintain predefined systolic and diastolic pressures within the cavity.

17. The device of claim 1, further comprising a central control system operably connected to the at least one pump, said central control system comprising a transmitter for transmitting data from the device.

18. The device of claim 9, further comprising a central control system operably connected to the pumps and the control valves, said central control system comprising a transmitter for transmitting data from the device.

19. The device of claim 6, further comprising a second supporting jacket configured for surrounding at least a portion of the right ventricle and a second expandable membrane attached to the second jacket so as to form a second inflatable cavity between the second jacket and the second membrane, said second membrane configured to face inwardly toward the right ventricle such that the second membrane contacts at least a portion of the right ventricle.

20. The device of claim 19, wherein each cavity is fluidly connected to the reservoir via a common line, and wherein the at least one pump is configured for pumping fluid back and forth between the reservoir and the cavities.
